(19) United States
(12) Patent Application Publication
(10) Pub. No.: US 2026/0308213 A1
(43) Pub. Date: Oct. 8, 2026

(54) METHOD FOR ISOLATING FETUIN-A

(71) Applicant: PreviPharma GmbH, Mannheim (DE)

(72) Inventors: Stephan T. KIESSIG, Wiesloch (DE); Rebecca Michaela REINIG, Heppenheim (DE); Tina BERGER, Bensheim (DE); Satish Kumar DEVARAPU, Mannheim (DE); Wolfgang TESCHNER, Wien (AT)

(21) Appl. No.: 19/484,066

(22) PCT Filed: May 13, 2024
(86) PCT No.: PCT/EP2024/063093

§ 371 Date: Nov. 13, 2025

(30) Foreign Application Priority Data
May 15, 2023 (EP) 23173323.9

(51) Int. Cl. C07K 14/47; A61K 38/00; A61P 19/10; B01D 15/38; B01D 61/14; G01N 33/68

(52) U.S. Cl. CPC C07K 14/473; A61P 19/10; B01D 15/3804; B01D 61/145; G01N 33/6893; A61K 38/00; G01N 2333/4728

(57) ABSTRACT

The present invention relates to a method for isolating Fetuin-A, comprising a step of contacting the fluid containing Fetuin-A to a solid phase that bears moieties of an anthraquinone compound, in particular Reactive Blue 2. Furthermore, the present invention refers a composition comprising Fetuin-A obtainable from such method and uses thereof. Moreover, the invention refers to a method for determining the activity of Fetuin-A.

The present invention relates to a method for isolating Fetuin-A, comprising a step of contacting a fluid containing Fetuin-A to a solid phase that bears moieties of an anthraquinone compound, in particular Reactive Blue 2. Furthermore, the present invention refers a composition comprising Fetuin-A obtainable from such method and uses thereof. Moreover, the invention refers to a method for determining the activity of Fetuin-A.

The invention provides a method for preparing Fetuin-A from a biological liquid. The method comprises the steps of providing a Fetuin-A containing source material, loading the Fetuin-A containing source material on an affinity chromatography material, wherein Fetuin-A binds to the affinity resin and elution conditions of the Fetuin-A.

Fetuin-A, also known as alpha-2-Heremans-Schmid glycoprotein, is a plasma protein, mainly synthesised in the liver and then secreted into the blood, but it is also produced in kidney and in choroid plexus (Price P A, Lim J E. The inhibition of calcium phosphate precipitation by fetuin is accompanied by the formation of a fetuin-mineral complex. J Biol Chem. 2003 Jun. 13; 278(24):22144-52). Due to its posttranslational modifications, such as glycosylation and phosphorylation, the protein has a molecular weight of 52 kDa. The modifications are thought to be important for protein expression, stability, and its biological activity, among other things. The physiological concentration of Fetuin-A in healthy humans is in the range of 0.3-1 g/L and has a half-life of several days. The protein has various functions in the human body such as protease inhibitor, it is known as an acute phase protein, it inhibits transforming growth factor β (TGF-β), and due to its high affinity for calcium or calcium salts, it is a protein for its transport in the blood (Galembeck F, Cann J R. Fetuin as a trypsin inhibitor. Arch Biochem Biophys. 1974 September; 164(1):326-31). Fetuin-A has a crucial function in mineral homeostasis and bone metabolism as in vitro demineralised bone was only remineralised in the presence of Fetuin-A, and Fetuin-A actively prevents calcification of soft tissue or blood vessels (Toroian D, Price P A. The essential role of fetuin in the serum-induced calcification of collagen. Calcif Tissue Int. 2008 February; 82(2):116-26). This is possible because Fetuin-A actively inhibits the precipitation of calcium-phosphate nanocrystals, which are produced during bone resorption, for example, by forming water-soluble Colloidal Calciprotein Particles (CPPs). CPPs are stabilised by Fetuin-A for up to 30 h and are transported to bone, where their calcium and phosphate are incorporated as ions into the collagen type I fibrils present there and contribute to bone mineralisation (Ketteler M, Bongartz P, Westenfeld R, Wildberger J E, Mahnken A H, Böhm R, Metzger T, Wanner C, Jahnen-Dechent W, Floege J. Association of low fetuin-A (AHSG) concentrations in serum with cardiovascular mortality in patients on dialysis: a cross-sectional study. Lancet. 2003 Mar. 8; 361(9360):827-33). It is therefore not surprising that Fetuin-A is one of the most abundant non-collagenous plasma proteins on bone.

The bone is a living, active tissue constantly remodelling itself through bone resorption and formation. An imbalance between the two stages leads to osteoporosis. Osteoporotic bones are spongier (porous) and have lower density or mass and contain abnormal tissue structure. Consequently, when bones become weak an increased risk of spontaneous fractures (hip, spine, or wrist) occurs. The basic framework of bones is given by collagen fibres. The minerals (calcium, phosphate) are responsible for hardening the bones. In both, women (5-7 years after menopause) and men osteoporosis onset with advanced age. Due to demographic changes, society is getting older and thus the number of patients with degenerative diseases is also increasing. In general, people with age of 50 years have a high risk of osteoporotic bone fractures. In 2010 158 million people worldwide suffered from osteoporosis, this number will be almost doubled up to 2040 to 310 million individuals (Sweet M G, Sweet J M, Jeremiah M P, Galazka S S. Diagnosis and treatment of osteoporosis. Am Fam Physician. 2009 Feb. 1; 79(3):193-200). 7-20 million people are expected in 2050 only with osteoporosis-related hip fractures. Twenty percent of the patients with hip fracture become functionally dependent and require long-term nursing care (Jaglal S B, Mclsaac W J, Hawker G, Carroll J, Jaakkimainen L, Cadarette S M, Cameron C, Davis D. Information needs in the management of osteoporosis in family practice: an illustration of the failure of the current guideline implementation process. Osteoporos Int. 2003 August; 14(8):672-6). The first symptoms of osteoporosis are permanent pain, losing height, stooped or hunched posture, limiting mobility often leading to isolation or depression.

The current standard of care is based on a basic therapy using calcium, phosphate, and vitamin D in combination with other drugs, which target the bone resorption by slowing medications (e.g., bisphosphonate, estrogen, or specific monoclonal antibodies acting on osteoclasts), or promote the bone building (e.g., fluorides, or parathyroid hormones) by increasing the activity of bone-building cells (osteoblasts). Nevertheless, the current therapy is not always sufficient to prevent vertebral and bone fractures. Moreover, there are severe side effects and potential adverse events on long-term use, and the treatment of osteoporosis and of related bone fractures is very cost intensive. In up to 25% of osteoporosis patients, treatment fails and they suffer bone fractures even though they are receiving medication for osteoporosis.

The exact mechanism of Fetuin-A in bone mineralization has not been completely elucidated, but during in vitro rat study, Toroian et al. noted that Fetuin-A stimulated calcification within bone (Toroian D, Price P A. The essential role of fetuin in the serum-induced calcification of collagen. Calcif Tissue Int. 2008 February; 82(2):116-26), but human studies on Fetuin-A to define its exact mechanism are limited (Ix J H, Wassel C L, Chertow G M, Koster A, Johnson K C, Tylavsky F A, Cauley J A, Cummings S R, Harris T B, Shlipak M G; Health Aging and Body Composition Study.

Fetuin-A and change in body composition in older persons. J Clin Endocrinol Metab. 2009 November; 94(11):4492-8) Fetuin-A is commercially available, especially prepared from foetal calf serum. It is known to prepare Fetuin-A by ammonium sulphate precipitation or by cold ethanol precipitation according to Spiro (Spiro R. G., Journal of Biological Chemistry 235, 10: 2860, 1960). Moreover, a one-step affinity purification of Fetuin from foetal bovine serum was developed on the basis of wheat germ agglutinin affinity separation (Cartellieri S. et al., Biotechnol. Appl. Biochem. 2002, 35 (2): 83-9). The preparation from foetal serum is widely preferred because the content of Fetuin-A in foetal plasma is much higher than in adult plasma. In addition, the preparation of pure and basically free of further impurities Fetuin-A from a biological liquid is described in EP3964523A1 which comprises the usage of hydrophobic interaction chromatography.

However, PreviPharma also developed another effective method, described in EP3865143A1 to absorb Fetuin-A from a biological liquid on a chelate chromatography material, wherein the material is charged with a doubly charged cation. The patent also describes the elution of very pure Fetuin-A.

Fetuin-A in plasma is often strongly bound to albumin in a way, that Fetuin-A can be prepared only as an Fetuin-A/Albumin complex. For pharmaceutical purposes, it would be preferable to provide an albumin-free Fetuin-A preparation. There is still a need for effective preparation methods for Fetuin-A from biological liquids, especially from human plasma, preferably from adult plasma or available fraction of human plasma, arising during the conventional plasma fractionation according to Cohn or Kistler-Nitschman. The present invention has the object to provide an efficient preparation method for Fetuin-A from biological liquids and especially from plasma or a plasma-derived preparation or from other sources. This object is solved by a method as depicted in the claims. Preferred embodiments are outlined in the dependent claims. Furthermore, the object is solved by a protein preparation and a pharmaceutical composition as depicted in the further independent and dependent claims.

Surprisingly, it has been found that Fetuin-A can be effectively isolated from fluids by using moieties of an anthraquinone compound, in particular Reactive Blue 2.

Furthermore, it has been found that the obtained Fetuin-A can be very well used for therapeutic purposes.

A first aspect of the present invention relates to a method for isolating Fetuin-A from a fluid containing Fetuin-A, wherein said method comprises the following steps:
(i) contacting the fluid containing Fetuin-A to a solid phase that bears moieties of an anthraquinone compound, in particular Reactive Blue 2;
(ii) allowing adhesion of the Fetuin-A to the solid phase;
(iii) at least partly removing the fluid from the solid phase and optionally washing the solid phase with a washing buffer; and
(iv) contacting the solid phase with an elution buffer and thereby eluting the Fetuin-A from the solid phase.

Surprisingly, it has been found that Fetuin-A can be very efficiently isolated and purified from biological fluids, even including separating it from albumin by using the method of the present invention. As found herein, an anthraquinone compound, in particular Reactive Blue 2, is unexpectedly useful for preparing Fetuin-A from fluids, in particular biological fluids, plasma, serum and plasma fractions.

Surprisingly the well-known binding albumin does not significantly interact with the selective binding of Fetuin-A. The isolation of Fetuin-A from a human plasma fraction by affinity chromatographic purification according to the present invention is experimentally shown.

Surprisingly, it has been found that the method of the present invention allows isolation of Fetuin-A in an essentially albumin-free preparation. Thus, it allows isolating Fetuin-A not only in a Fetuin-A/albumin complex, but as pure Fetuin-A. It was found that the resulting Fetuin-A preparations are highly pure and basically free of further impurities, especially essentially free from albumin although albumin may also bind to the solid material with comparably high affinity. A major advantage of the method of the present invention is the effective separation of albumin from the preparation. Surprisingly, Fetuin-A elutes at higher salt concentrations, although solid phases that bear moieties of an anthraquinone compound, in particular Reactive Blue 2, are rather known to not bind other plasma proteins or elute them with relatively low salt conditions.

In a preferred embodiment, the present invention relates to preparing/isolating Fetuin-A from a Fetuin-A-containing source material, e.g., a biological fluid, comprising the following steps:
providing a Fetuin-A-containing source material;
transferring the biological fluid into a buffer that enables positive affinity chromatographic purification;
loading the buffered Fetuin-A containing source material on the affinity interaction chromatography material, wherein Fetuin-A adsorbs to the chromatography material, and eluting Fetuin-A.

In the context of the present invention, Fetuin-A may be understood in the broadest sense as generally understood in the art. In the context of the present invention, the terms “Fetuin-A”, “fetuin-A”, Fetuin-A”, “Fetuin-A”, “alpha2-HS glycoprotein”, “α2-HS glycoprotein”, “AHSG”, “Ahsg”, “α2-HS”, “A2HS”, “AHS” and “HSGA” may be understood interchangeably.

The person skilled in the art knows Fetuin-A. Fetuin-A may be of any species. Preferably, Fetuin-A is mammalian or bovine Fetuin-A. Preferably, Fetuin-A is human, mouse, rat, or bovine Fetuin-A. Preferably, Fetuin-A is human Fetuin-A.

As used herein, “isolating” may be understood interchangeably with “purifying” in the broadest sense as increasing the content of the isolated Fetuin-A in a composition. It does not necessarily be pure. However, preferably, the product of the method comprises at least 25% (w/w), based on the total protein weight, of Fetuin-A. More preferably, the product of the process comprises at least 50% (w/w), even more preferably at least 70% (w/w), even more preferably at least 80% (w/w), in particular at least 90% (w/w), based on the total protein weight, of Fetuin-A.

Anthraquinone compounds, which are usable for binding proteins, are known in the art.

In a preferred embodiment, the anthraquinone compound is an anthraquinone-based compound that is binding Fetuin-A, preferably with a dissociation constant Kd of less than 10⁻² M, in particular of less than 10⁻⁴ M or less than 10⁻⁵ M.

In a preferred embodiment, the anthraquinone compound is an anthraquinone-based compound that is binding albumin, preferably human serum albumin, preferably with a dissociation constant Kd of less than 10⁻² M, in particular of less than 10⁻⁴ M or less than 10⁻⁵ M.

In a preferred embodiment, the anthraquinone compound is an anthraquinone dye either modified or unmodified. In a preferred embodiment, the anthraquinone compound is selected from the group consisting of Reactive Blue 2, an alizarin dye, carminic acid dye (e.g., E120), kermesic acid dye, laccainic acid dye, 1,4-anthraquinone, Remazol Brillant Blue R, Procion Blue MX-R, and 1-amino-4,8-die-HO-5-Ph-amino-4A,9A-2H-anthraquinone.

In a preferred embodiment, the anthraquinone compound is selected from the group consisting of Reactive Blue 2. It may be understood as the compound Reactive Blue 2 may be designated by CAS No. 12236-82-7 or a salt thereof. Reactive Blue 2 may also be designated chemically as 1-amino-4-[4-[[4-chloro-6-(3-sulfoanilino)-1,3,5-triazin-2-yl]amino]-3-sulfoanilino]-9,10-dioxoanthracene-2-sulfonic acid or as 1-amino-4-{[4-({4-chlor-6-[(3-sulfophenyl)amino]-1,3,5-triazin-2-yl}amino)-3-sulfophenyl]amino}-9,10-dioxo-9,10-dihydro-2-sulfonic acid.

It will be understood that Reactive Blue 2 in the sense of the present invention also embraces the salts thereof. As used herein, the anthraquinone compound Reactive Blue 2 may also be designated as “Affi gel blue”, “Affi-Gel Blue Gel column”, “Affi-gel blue”, “Basilen Blue”, “Cibacron Blue 3G-A”, “Cibacron Blue 3G”, “Cibacron Blue F 3GA”, “Cibacronblau F3G-A”, “Cibacron Blue F(3)GA”, “Cibacron blue F3G-A”, “CHEBI:34946”, “C.I. 61211”, “Protein Blue H-B” “Procion Blue H-B”, or “Procion Blue HB”.

An anthraquinone compound, in particular Reactive Blue 2, may bear functions such as an ionic, hydrophobic, aromatic, and/or sterically active binding sites. Solid material bearing an anthraquinone compound, in particular Reactive Blue 2, moiety is commercially available. It has been described for the binding of albumin.

The fluid containing Fetuin-A may be any fluid. In a preferred embodiment, the fluid is a body fluid. In a preferred embodiment, the fluid is blood, blood serum, blood plasma or a fraction thereof.

In a preferred embodiment, the fluid is prepared from a blood plasma fraction selected from the group consisting of:
(a) crude V supernatant;
(b) paste IV or a subfraction thereof of the Cohn process;
(c) paste IV or a subfraction thereof of the Kistler-Nitschmann process;
(d) a waste fraction of paste I+II+III or I+III of the Cohn process or a combination thereof;
(e) a waste fraction of paste I+II+III or I+III of the Kistler-Nitschmann process or a combination thereof;
(f) paste I+II+III or paste I+III of the Cohn process or any fraction or a combination thereof;
(g) cryo-poor plasma supernatant or cryo-poor plasma precipitate;
(h) paste I+II+III or paste I+III of the Kistler-Nitschmann process or any fraction or a combination thereof; and
(i) a combination of two or more thereof.

Subfraction of fraction IV may for example be subfraction IV.1 and/or subfraction IV.4. In a preferred embodiment, the fluid is crude V supernatant.

The solid material may be any solid material to which an anthraquinone compound, in particular Reactive Blue 2, is conjugated to. In other words, an anthraquinone compound, in particular Reactive Blue 2, is immobilized on the solid phase. In other words, an anthraquinone compound, in particular Reactive Blue 2, is bound on the solid phase, in particular bound covalently to the solid phase. It will be understood that the material will typically not disturb the polypeptide structure of Fetuin-A.

In a preferred embodiment, the solid phase is an affinity chromatography material. In a preferred embodiment, the solid phase comprises or consists of spherical beads of an affinity chromatography material.

The solid phase may be contacted with the fluid containing Fetuin-A by any means. For instance, it may be admixed with the fluid or the fluid is added to the solid phase. The solid phase may also be designated as a resin or gel such as a chromatography resin or a chromatography gel.

When adding the fluid to the solid phase, it may be optionally previously admixed with one or more other liquids, in particular may be optionally admixed with a loading buffer. The loading step may be performed under conditions where Fetuin-A adheres to the solid material. As used throughout the present invention, a buffer is understood as an aqueous solution and may thus also be designatable as “aqueous buffer”.

In the context of the present invention, any buffer that does not prevent the adhesion of the Fetuin-A to the solid material may be used as loading buffer. In a preferred embodiment, a loading buffer may be a buffer containing not more than 0.1 M of cations. In a preferred embodiment, a loading buffer may be a buffer of pH 6 to 9 containing not more than 0.1 M of cations. In a preferred embodiment, a loading buffer may be a buffer of pH 6 to 8 containing not more than 0.1 M of cations. In a preferred embodiment, a loading buffer may be a 0.01 to 0.1 M sodium phosphate buffer, 0.005 to 0.1 M sodium phosphate buffer, 0.007 to 0.08 M sodium phosphate buffer, 0.01 to 0.05 M sodium phosphate buffer, 0.015 to 0.025 M sodium phosphate buffer, or approximately a 0.015 to 0.025 M sodium phosphate buffer.

In a preferred embodiment, a loading buffer may be a buffer of pH 6.0 to 8.5, a buffer of pH 6.5 to 8.0, a buffer of pH 6.8 to 7.6, a buffer of pH 6.9 to 7.5, a buffer of pH 7.0 to 7.4, or a buffer of approximately pH 7.1. In a preferred embodiment, a loading buffer may be a 0.01 to 0.1 M sodium phosphate buffer of pH 6.0 to 8.5. In a preferred embodiment, the loading buffer bears the same characteristics as the washing buffer as used in the method. In a preferred embodiment, the step is performed at a pH in the range of 6 to 8, more preferably at approximately pH 7.

In a preferred embodiment, the solid phase is placed in a chromatography column allowing the flow through of fluids.

In a preferred embodiment, the solid phase placed in a chromatography column is equilibrated by the loading or washing buffer. In a preferred embodiment, the solid phase placed in a chromatography column is equilibrated by the loading or washing buffer and the fluid containing Fetuin-A (as such or admixed with a loading buffer) is applied to the solid material, preferably by a liquid flow.

The solid phase may be any solid material that bears an anthraquinone compound, in particular Reactive Blue 2. It will be understood that it should typically not prevent the adhesion of Fetuin-A to an anthraquinone compound, in particular Reactive Blue 2.

In a preferred embodiment, the solid phase is characterized in that an anthraquinone compound, in particular Reactive Blue 2, is covalently conjugated with a solid material. The solid material may be any solid material that does not prevent the adhesion of Fetuin-A to an anthraquinone compound, in particular Reactive Blue 2. In a preferred embodiment, the solid material is a solid material selected from the group consisting of a polysaccharide, a synthetic polymer, silica, and a combination of two or more thereof. In a preferred embodiment, the solid material is a solid material selected from the group consisting of agarose, in particular in Sepharose, acrylate, latex, and a combination of two or more thereof. In a preferred embodiment, the solid material is a Sepharose, i.e., a bead form of agarose.

Respective solid phases usable for chromatographic purposes that comprise moieties of an anthraquinone compound, in particular Reactive Blue 2, are commercially available such a, e.g., Affi-Gel Blue Gel (BioRad, Germany).

The step of allowing adhesion of the Fetuin-A to the solid phase may be achieved by any means allowing the interaction of the Fetuin-A with the solid phase, in particular an anthraquinone compound, in particular Reactive Blue 2, bound to the solid phase. Thus, in a preferred embodiment, the salt concentration is low enough to enable adherence in particular below 0.1 M (related to cations). In a preferred embodiment, the temperature is suitable for adherence, preferably is below 45° C., below 40° C., between 0 and 40° C., between 1 and 38° C., between 1 and 5° C., or between 5° C. and 30° C.

Adherence may also be understood as adsorption or interaction. Preferably, the adherence of Fetuin-A to the solid material, in particular an anthraquinone compound, in particular Reactive Blue 2, is not covalent.

The step of at least partly removing the fluid from the solid phase to which Fetuin-A adheres may be achieved by any means such as by removing the fluid by replacing it with other liquid such as a washing buffer and/or sucking it from the material, for instance, in a column. Alternatively, the solid phase to which Fetuin-A adheres may be separated from the liquid by any other means such as sedimentation, centrifugation, and/or filtration.

Optionally, the solid phase to which Fetuin-A adheres is washed. Such optional step of washing may be achieved by any means such as by flowing washing buffer along the solid phase to which Fetuin-A adheres, for instance, in a column. Alternatively, the solid phase to which Fetuin-A adheres may be separated from the washing buffer by any other means such as sedimentation, centrifugation, and/or filtration.

In the context of the present invention, any buffer that does not disturb the adhesion of the Fetuin-A to the solid material may be used as washing buffer. In a preferred embodiment, the washing buffer is a buffer containing not more than 0.1 M of cations. In a preferred embodiment, the washing buffer is a buffer of pH 6 to 9 containing not more than 0.1 M of cations. In a preferred embodiment, the washing buffer is a buffer of pH 6 to 8 containing not more than 0.1 M of cations.

In a preferred embodiment, the washing buffer may be a 0.01 to 0.1 M sodium phosphate buffer, 0.005 to 0.1 M sodium phosphate buffer, 0.007 to 0.08 M sodium phosphate buffer, 0.01 to 0.05 M sodium phosphate buffer, 0.015 to 0.025 M sodium phosphate buffer, a 0.007 to 0.08 M sodium phosphate buffer, a 0.01 to 0.05 M sodium phosphate buffer, a 0.015 to 0.025 M sodium phosphate buffer, or approximately a 0.015 to 0.025 M sodium phosphate buffer. In a preferred embodiment, the washing buffer may be a buffer of pH 6.0 to 8.5, a buffer of pH 6.5 to 8.0, a buffer of pH 6.8 to 7.6, a buffer of pH 6.9 to 7.5, a buffer of pH 7.0 to 7.4, or a buffer of approximately pH 7.1. In a preferred embodiment, the washing buffer may be a 0.01 to 0.1 M sodium phosphate buffer of pH 6.0 to 8.5. In a preferred embodiment, the washing buffer bears the same characteristics as the loading buffer as far as used. In a preferred embodiment, the step is performed at a pH in the range of 6 to 8, more preferably at approximately pH 7.

The adhesion of the Fetuin-A to an anthraquinone compound, in particular Reactive Blue 2, may be achieved by any means. In a preferred embodiment, the adhesion is reversible, preferably is not covalent.

After a washing step the elution may be performed, wherein a gradient was set to increase a salt concentration (e.g., NaCl concentration) to weaken the interaction of Fetuin-A and the solid phase. Without being bound to this theory, the elution of the adhered Fetuin-A may be performed by increased ionic strength.

In the context of the present invention, any buffer that allows the elution (also: detachment) of the Fetuin-A from the solid material may be used as elution buffer.

In a preferred embodiment, the elution buffer does not destroy the three-dimensional structure of Fetuin-A, in particularly does not irreversibly destroy the three-dimensional structure of Fetuin-A.

The step (iii) of at least partly removing the fluid from the solid phase and optionally washing the solid phase with a washing buffer, may also inherently include the removal of agents using in preceding treatment of the fluid containing Fetuin-A such as, e.g., precipitation agents usable for obtaining blood plasma fractions (e.g., ethanol).

At increasing salt concentrations, Fetuin-A may be eluted from the solid phase. Increasing salt concentrations (e.g., NaCl concentrations) may be performed by any means and in any gradient.

For example, the content ratio of a high-salt elution buffer may be increasingly admixed. Such gradient may be a linear gradient. It may also have any other profile.

In a preferred embodiment, the elution buffer is a buffer containing at least 0.5 M of cations. In a preferred embodiment, the elution buffer is a buffer containing at least 0.6 M of cations, at least 0.7 M of cations, at least 0.8 M of cations, at least 0.9 M of cations, at least 1.0 M of cations, at least 1.1 M of cations, or at least 1.2 M of cations, at least 1.3 M of cations. In a preferred embodiment, the elution buffer is a buffer containing 0.5 to 5.0 M of cations, or 0.5 to 2.0 M of cations, or 0.7 to 1.8 M of cations, or 1.0 to 1.6 M of cations, or 1.3 to 1.5 M of cations, or approximately a 1.4 M of cations.

In a preferred embodiment, the elution buffer is a buffer containing at least 0.5 M of sodium chloride. In a preferred embodiment, the elution buffer is a buffer containing at least 0.6 M of sodium chloride, at least 0.7 M of sodium chloride, at least 0.8 M of sodium chloride, at least 0.9 M of sodium chloride, at least 1.0 M of sodium chloride, at least 1.1 M of sodium chloride, or at least 1.2 M of sodium chloride, at least 1.3 M of sodium chloride. In a preferred embodiment, the elution buffer is a buffer containing 0.5 to 5.0 M of sodium chloride, or 0.5 to 2.0 M of sodium chloride, or 0.7 to 1.8 M of sodium chloride, or 1.0 to 1.6 M of sodium chloride, or 1.3 to 1.5 M of sodium chloride, or approximately 1.4 M of sodium chloride.

In a preferred embodiment, the elution buffer may be a buffer of pH 6.0 to 8.5, a buffer of pH 6.5 to 8.0, a buffer of pH 6.8 to 7.6, a buffer of pH 6.9 to 7.5, a buffer of pH 7.0 to 7.4, or a buffer of approximately pH 7.1.

In a preferred embodiment, the elution buffer has a molarity of about 1.4 M NaCl, in 20 mM phosphate buffer. In a preferred embodiment, a NaCl gradient may be used for elution.

In a preferred embodiment, the elution buffer is a buffer of pH 6 to 9 containing more than 0.5 M of cations.

In a preferred embodiment, the elution buffer is a buffer of pH 6 to 8 containing more than 0.5 M of cations. In a preferred embodiment, the elution buffer may be a phosphate buffer.

In a preferred embodiment, the elution buffer may comprise 0.01 to 0.1 M sodium phosphate, 0.007 to 0.08 M sodium phosphate, 0.01 to 0.05 M sodium phosphate buffer, 0.015 to 0.025 M sodium phosphate, or approximately 0.015 to 0.025 M sodium phosphate. In a preferred embodiment, the step is performed at a pH in the range of 6 to 8, more preferably at approximately pH 7.

In a preferred embodiment, the elution buffer is a 0.01 to 0.1 M sodium phosphate buffer of pH 6.0 to 8.5 containing 0.5 to 2.0 M sodium chloride.

Optionally, the method may further comprise additional steps for processing Fetuin-A and compositions comprising such.

In a preferred embodiment, the method further one or more additional chromatographic steps, preferably one or more additional chromatographic steps selected from the group consisting of cation exchange chromatography, size-exclusion chromatography, anion exchange chromatography, hydrophobic interaction chromatography, and combinations of two or more thereof.

In a preferred embodiment, the method further comprises one or more of the following steps:
(a) subjecting a solution containing Fetuin-A to ultrafiltration/diafiltration, nanofiltration, a combination of ultrafiltration and nanofiltration, or any other virus removal procedure;
(b) a step of adjusting the pH of a solution containing the eluted Fetuin-A;
(c) a step of stabilizing the Fetuin-A by adding one or more stabilizers that prevent the Fetuin-A from degradation;
(d) one or more antiviral treatments, like nanofiltration, solvent/detergent treatment, pasteurization or dry-heat treatment
(e) adding one or more detergents, preferably one or more detergents selected from the group consisting of Tween-20, Tween-80, BRIJ-35 (polyoxyethylen(23)laurylether), Brji-58 (polyethylene glycol hexadecyl ether, polyoxyethylene (20) cetyl ether), NP-40 (octyl-phenoxy(polyoxyethylene)ethano), Triton-X-100, IGEPAL (octyl-phenoxy(polyoxyethylene)ethanol) Tergitol (nonyl-phenyl-polyethylene glycol), and derivatives thereof;
(f) adding one or more other antiviral agents such as a phosphate ester, in particular tri-n-butyl-phosphate; or
(g) dialysis; or
(h) a combination of two or more thereof.

In a preferred embodiment, the method may comprise one or more antiviral treatments, preferably including adding one or more other antiviral agents such as a phosphate ester, in particular tri-n-butyl-phosphate (TNBP), solvent/detergent treatment, UV irradiation, nanofiltration, heat treatment, dry heat treatment, and/or others.

In a preferred embodiment, the method may comprise drying or freeze drying Fetuin-A.

Filtration, including ultrafiltration and nanofiltration, may include dead-end ultrafiltration, tangential flow ultrafiltration, or a combination thereof. In a preferred embodiment, the method comprises tangential flow filtration (e.g., with Pall T-Serie TFF Cassette with Omega™ Membrane 200 cm² 30 kDa).

Optionally, the obtained Fetuin-A may be analyzed by any means. For instance, it may be analyzed by SDS-PAGE and/or Western Blotting. Here, it may result in a band at approximately 52 kDa.

As indicated above, it was surprisingly found that an anthraquinone compound, in particular Reactive Blue 2, may be used for isolating Fetuin-A.

In a preferred embodiment, the method of the present invention comprises:
providing blood plasma or a plasma fraction comprising Fetuin-A, preferably crude V supernatant (optionally including ethanol removal);
equilibrating the chromatography material;
loading the blood plasma or plasma fraction on chromatography column, and adsorbing Fetuin-A to the material;
washing the adsorbed protein with wash buffer; and
eluting Fetuin-A with the elution buffer, wherein the cations may be applied by a salt gradient, preferably a NaCl gradient (e.g., using a concentration up to a maximal concentration, e.g., 1.4 M NaCl).

The molarity in the solution comprising Fetuin-A as obtained from the elution step may optionally be adjusted to a desired range or value.

The solution comprising Fetuin-A as obtained from the elution step may or may not comprise albumin. The solution comprising Fetuin-A as obtained from the elution step may or may not comprise transferrin. The solution comprising Fetuin-A as obtained from the elution step may or may not comprise apolipoprotein A-II. The solution comprising Fetuin-A as obtained from the elution step may or may not comprise one or more other proteins different from Fetuin-A, albumin, transferrin, and apolipoprotein A-II.

In a preferred embodiment, the solution comprising Fetuin-A as obtained from the elution step does not comprise more than 50% by weight, does not comprise more than 25% by weight, does not comprise more than 10% by weight, does not comprise more than 5% by weight, does not comprise more than 2% by weight, does not comprise more than 1% by weight, does not comprise more than 0.5% by weight, or does not comprise more than 0.1% by weight, based on the total protein content of the solution, of albumin or transferrin.

In a preferred embodiment, the solution comprising Fetuin-A as obtained from the elution step does not comprise more than 50% by weight, does not comprise more than 25% by weight, does not comprise more than 10% by weight, does not comprise more than 5% by weight, does not comprise more than 2% by weight, does not comprise more than 1% by weight, does not comprise more than 0.5% by weight, or does not comprise more than 0.1% by weight, based on the total protein content of the solution, of apolipoprotein A-II.

In a preferred embodiment, the solution comprising Fetuin-A as obtained from the elution step does not comprise more than 50% by weight, does not comprise more than 25% by weight, does not comprise more than 10% by weight, does not comprise more than 5% by weight, does not comprise more than 2% by weight, does not comprise more than 1% by weight, does not comprise more than 0.5% by weight, or does not comprise more than 0.1% by weight, based on the total protein content of the solution, of a total amount of other proteins different from Fetuin-A, albumin, transferrin, and apolipoprotein A-II.

In a preferred embodiment, the solution comprising Fetuin-A as obtained from the elution step does not comprise more than 50% by weight, does not comprise more than 25% by weight, does not comprise more than 10% by weight, does not comprise more than 5% by weight, does not comprise more than 2% by weight, does not comprise more than 1% by weight, does not comprise more than 0.5% by weight, or does not comprise more than 0.1% by weight, based on the total protein content of the solution, of a sum of albumin, transferrin, and apolipoprotein A-II.

Accordingly, a further aspect of the present invention relates to the use of a solid phase that bears moieties of an anthraquinone compound, in particular Reactive Blue 2, for isolating Fetuin-A.

It will be understood that the embodiments and definitions as laid out in the context of the method above mutatis mutandis apply to the use. In a preferred embodiment, the use comprises one or more of the method steps described herein.

The present invention relates to compositions comprising Fetuin-A in particularly high content and beneficial characteristics.

Thus, a further aspect of the present invention also refers to a composition comprising Fetuin-A obtainable from the method of the present invention, wherein the composition comprises at least 50% by weight, based on the total protein mass, of Fetuin-A.

It will be understood that the embodiments and definitions as laid out in the context of the method above mutatis mutandis apply to the composition.

Composition may be stored at any conditions suitable for such purpose. The storage form may be in liquid form, but may also be a dried form (e.g., a powder such as a powder comprising dried or freeze-dried Fetuin-A) or may be a paste or syrup or the like. Optionally, a dried form, paste or syrup may be dissolved or emulsified prior to use thereof, such as, e.g., for being administered to a patient. In a preferred embodiment, Fetuin-A is stored in a solution of pH 6 to 8, of pH 6.5 to 7.5, of pH 6.9 to 7.4, or approximately pH 7.1 or approximately pH 7.2. In a preferred embodiment, Fetuin-A is stored in a PBS buffer, preferably a PBS buffer of pH 6 to 8, of pH 6.5 to 7.5, of pH 6.9 to 7.4, or approximately pH 7.1 or approximately pH 7.2.

Such storage may be optionally long-term storage such as, e.g., for at least one week, for at least one month, for at least six months, or for at least one year.

The composition of the present invention may or may not comprise albumin. The composition of the present invention may or may not comprise transferrin. The composition of the present invention may or may not comprise apolipoprotein A-II. The composition of the present invention may or may not comprise one or more other proteins different from Fetuin-A, albumin, transferrin, and apolipoprotein A-II.

In a preferred embodiment, the composition of the present invention does not comprise more than 50% by weight, does not comprise more than 25% by weight, does not comprise more than 10% by weight, does not comprise more than 5% by weight, does not comprise more than 2% by weight, does not comprise more than 1% by weight, does not comprise more than 0.5% by weight, or does not comprise more than 0.1% by weight, based on the total protein content of the solution, of albumin.

In a preferred embodiment, the composition of the present invention does not comprise more than 50% by weight, does not comprise more than 25% by weight, does not comprise more than 10% by weight, does not comprise more than 5% by weight, does not comprise more than 2% by weight, does not comprise more than 1% by weight, does not comprise more than 0.5% by weight, or does not comprise more than 0.1% by weight, based on the total protein content of the solution, of transferrin.

In a preferred embodiment, the composition of the present invention does not comprise more than 50% by weight, does not comprise more than 25% by weight, does not comprise more than 10% by weight, does not comprise more than 5% by weight, does not comprise more than 2% by weight, does not comprise more than 1% by weight, does not comprise more than 0.5% by weight, or does not comprise more than 0.1% by weight, based on the total protein content of the solution, of apolipoprotein A-II.

In a preferred embodiment, the composition of the present invention does not comprise more than 50% by weight, does not comprise more than 25% by weight, does not comprise more than 10% by weight, does not comprise more than 5% by weight, does not comprise more than 2% by weight, does not comprise more than 1% by weight, does not comprise more than 0.5% by weight, or does not comprise more than 0.1% by weight, based on the total protein content of the solution, of a total amount of other proteins different from Fetuin-A, albumin, transferrin, and apolipoprotein A-II.

In a preferred embodiment, the composition of the present invention does not comprise more than 50% by weight, does not comprise more than 25% by weight, does not comprise more than 10% by weight, does not comprise more than 5% by weight, does not comprise more than 2% by weight, does not comprise more than 1% by weight, does not comprise more than 0.5% by weight, or does not comprise more than 0.1% by weight, based on the total protein content of the solution, of a sum of albumin, transferrin, and apolipoprotein A-II.

In a preferred embodiment, the composition comprises at least 60% by weight, at least 70% by weight, at least 80% by weight, or at least 90% by weight, or at least 95% by weight based on the total protein mass, of a sum of Fetuin-A, albumin, transferrin and apolipoprotein A-II.

In a preferred embodiment, the composition comprises at least 60% by weight, at least 70% by weight, at least 80% by weight, or at least 90% by weight, or at least 95% by weight based on the total protein mass, of Fetuin-A.

Optionally, the composition may comprise one or more further ingredients such as a as liquid carrier (e.g., a buffer). Optionally, the composition may be a pharmaceutical composition. Moreover, the invention comprises pharmaceutical compositions comprising a protein preparation comprising Fetuin-A as described above and at least one pharmaceutically acceptable carrier for use in treatment or prophylaxis in patients suffering from Fetuin-A deficiency.

As used herein, the terms “pharmaceutical composition” and “pharmaceutical formulation” may be understood interchangeably. As used herein, the terms “pharmaceutically acceptable carrier”, “pharmaceutically acceptable excipient”, “carrier” and “excipient” may be understood interchangeably in the broadest sense as any substance that may support or at least not prevent the pharmacological acceptance of the Fetuin-A. Such pharmaceutical composition may be ready to use and may preferably be a liquid formulation, in particular an injection portion.

A pharmaceutically acceptable carrier may exemplarily be selected from the list consisting of an aqueous buffer, saline, water, dimethyl sulfoxide (DMSO), ethanol, vegetable oil, paraffin oil or combinations of two or more thereof. Furthermore, the pharmaceutically acceptable carrier may optionally contain one or more detergent(s), one or more foaming agent(s) (e.g., sodium lauryl sulfate (SLS), sodium dodecyl sulfate (SDS)), one or more coloring agent(s) (e.g., food coloring), one or more vitamin(s), one or more salt(s) (e.g., sodium, potassium, calcium, zinc salts), one or more humectant(s) (e.g., sorbitol, glycerol, mannitol, propylene glycol, polydextrose), one or more enzyme(s), one or more preserving agent(s) (e.g., benzoic acid, methylparaben, one or more antioxidant(s), one or more herbal and plant extract(s), one or more stabilizing agent(s), one or more chelating agents (e.g., ethylenediaminetetraacetic acid (EDTA), and/or one or more uptake mediator(s) (e.g., polyethylene imine (PEI), a cell-penetrating peptide, a protein transduction domain (PTD), an antimicrobial peptide, etc.).

The present invention also relates to a dosage unit of the pharmaceutical composition usable in the context of the treatment or prevention of the present invention. Exemplarily, the present invention may refer to a single dose container or to a multiple dosage form.

The composition of the present invention is also suitable for being used in a medical/therapeutic context.

Accordingly, a further aspect of the present invention relates to Fetuin-A, preferably Fetuin-A obtainable from the method of the present invention, or a composition comprising Fetuin-A of the present invention, for use as a medicament.

For instance, a Fetuin-A deficiency may be treated. Generally, Fetuin-A deficiency may be independent from the cause of the deficiency, namely inborn or acquired Fetuin-A deficiency. Patients suffering from Fetuin-A deficiency may be treated or prophylactically treated with the pharmaceutical composition comprising Fetuin-A. Thus, Fetuin-A according to the invention may be used also to treat patients suffering from acquired or inborn Fetuin-A deficiencies.

Those patients may therefore be at risk to develop an osteoporosis with a high risk for bone fractures. In addition, Fetuin-A may be used also as a treatment of patients suffering of Osteogenesis imperfecta, as Fetuin-A is of interest for calcium and phosphate transport in plasma to the bone. Furthermore, Fetuin-A is known to prevent the calcification of soft tissues that occurs with age in people with Hutchinson-Gilford syndrome. Therefore, Fetuin-A may be used in a method for treating these patients and prevent further impairment due to calcification of tissues and heart caused by this disease.

The multiple mechanisms of action of Fetuin-A demonstrate the interest of Fetuin-A for the human body. For optimal compatibility, it is desirable to obtain Fetuin-A from human plasma. Since plasma fractions vary widely in composition, various purification methods for Fetuin-A from human biological fluids may be of interest to make the most effective and complete use of available resources.

A further aspect of the present invention relates to Fetuin-A, preferably Fetuin-A obtainable from the method of the present invention, or a composition comprising Fetuin-A of the present invention for use in a method for treating or preventing osteoporosis or being at risk bone fractures, a risk for hypercalcification (calciphylaxis).

It will be understood that the embodiments and definitions as laid out in the context of the method and the composition above mutatis mutandis apply to its use as a medicament and for use in therapeutic treatments. In other words, the present invention relates to a method for treating or preventing osteoporosis or being at risk bone fractures, a risk for hypercalcification (calciphylaxis), wherein said method comprises administration of a sufficient amount of Fetuin-A, preferably Fetuin-A obtainable from the method of the present invention, or a composition comprising Fetuin-A of the present invention to a patient in need thereof.

As used in the context of the present invention, the term “patient” may be understood in the broadest sense as a subject the Fetuin-A is administered to, irrespective, whether it is a human or an animal and whether clinical symptoms occur or do not occur. Preferably, the patient is a human patient.

The Fetuin-A may be administered to the patient by any means. Preferably, the polypeptide is injected into the tissue or into a blood vessel via a syringe or a drip. Alternatively, it may also be injected intraperitoneally, or it may be administered orally, nasally, topically or subcutaneously. Exemplarily, the Fetuin-A may be injected intravenously (i.v.), intraperitoneally (i.p.), intraarterially (i.a.), intramuscularly (i.m.) and/or subcutaneously (s.c.). Alternatively, the Fetuin-A may be taken up orally, e.g., as a powder, a tablet, a pill, a capsule, a chewable capsule, syrup, juice, gel, liquid or paste. Alternatively, Fetuin-A may be taken up nasally (intra nasal) (e.g., as spray or aerosol), percutaneously (e.g., as cream, spray or ointment and/or via a coated plaster) and/or by inhalation (e.g., inhalation of an aerosol or of a spray). It will be understood that the Fetuin-A may be administered locally or systemically.

The present invention further relates to means for showing the preserved activity of Fetuin-A. A further aspect of the present invention thus refers to a method for determining the activity of Fetuin-A, preferably Fetuin-A obtained from a method of the present invention. A supersaturated calcium phosphate solution can be mixed and incubated with protein solutions containing Fetuin-A of the present invention, commercially available Fetuin-A, albumin or any other protein at 37° C. The potential elongation of CPP formation over time caused by the protein used for the assay may be measured by optical density.

A further aspect relates to a method for determining the activity of Fetuin-A, preferably Fetuin-A obtained from a method of any one of claims 1 to 9, said method comprising the following steps:
(i) providing a solution containing:
   (a) a first solution containing soluble calcium salt,
   (b) a second solution containing a soluble phosphate salt, and
   (c) a sample containing the Fetuin-A;
(ii) mixing the components provided in step (i) with each other; and
(iii) determining the precipitation time of insoluble calcium phosphate,

wherein an elongated precipitation time indicates activity of Fetuin-A.

It will be understood that the embodiments and definitions as laid out in the context of the method, compositions, use as a medicament and for use in therapeutic treatments above mutatis mutandis apply to the method for determining the activity of Fetuin-A.

The method may be conducted at any temperature. In a preferred embodiment, the method is conducted at a temperature of 0 to 40° C., preferably at a temperature of 4 to 30° C., in particular at a temperature of 18 to 25° C. (i.e., for example, at ambient temperature).

The method may be conducted at solution. In a preferred embodiment, the method is conducted in aqueous buffers. In a preferred embodiment, the method is conducted in aqueous buffers at a pH in the range of 1 to 10, preferably at a pH in the range of 5 to 8, in particular at a pH of 6.8 to 7.6.

The soluble calcium salt may be any soluble calcium salt such as, e.g., CaCl₂. In a preferred embodiment, the first buffer is an aqueous solution. In a preferred embodiment, the first buffer is an aqueous buffer of a pH in the range of 1 to 10, preferably a pH in the range of 5 to 8, in particular a pH of 6.8 to 7.6. In a preferred embodiment, the first buffer contains between 1 and 200 mM of calcium ions, preferably 10 and 100 mM of calcium ions, in particular 20 and 60 mM of calcium ions.

The soluble phosphate salt may be any soluble phosphate salt such as, e.g., an alkali salt of hydrogen phosphate, an alkali salt of dihydrogen phosphate, or a mixture thereof (e.g., Na₂HPO₄, NaH₂PO₄, or a mixture of Na₂HPO₄ and NaH₂PO₄).

In a preferred embodiment, the second buffer is an aqueous solution. In a preferred embodiment, the second buffer is an aqueous buffer of a pH in the range of 1 to 10, preferably a pH in the range of 5 to 8, in particular a pH of 6.8 to 7.6. In a preferred embodiment, the second buffer contains between 1 and 200 mM of phosphate ions, preferably 5 and 100 mM of phosphate ions, in particular 10 and 40 mM of phosphate ions.

The test may be used for any concentration range of Fetuin-A such as, e.g., 0.001 to 10 gL, preferably 0.01 to 1 g/L, in particular 0.05 to 0.5 g/L.

In a preferred embodiment, an increase of delay of precipitation may indicate increasing Fetuin-A activity. This may, for instance, be an increase over a control sample without addition of Fetuin-A (e.g., another body fluid sample (e.g., another serum fraction) (essentially) not containing Fetuin-A).

The following examples and figures and claims are intended to provide illustrative embodiments of the present invention described and claimed herein. The examples and figures are not intended to provide any limitation on the scope of the invented subject-matter.

### BRIEF DESCRIPTION OF THE FIGURES

FIG. 1 shows a chromatogram of Fetuin-A purification from Crude 5 supernatant using chromatographic purification with Affi-Gel Blue Gel column (Affi blue media, Bio-Rad Laboratories GmbH, Feldkirchen, Germany). The purification included the equilibration of the column with water and loading buffer, the sample application, a wash step, followed by a salt gradient up to 1.4 M NaCl and a CIP step. The solid line shows the UV absorption at 260 nm. The dashed line depicts the salt gradient (in % by weight with a top at 1.4 M NaCl). The dashed-dotted line depicts the conductivity (in mS/cm).

FIG. 2 shows an SDS-PAGE performed to determine the purity and composition of the chromatographic Fetuin-A purification fractions. Fetuin-A was detected in the Load (L), flow through (FT), elution fractions (A) and in a low amount in the strip fractions (S). Fetuin-A standard (FetA), albumin standard (Alb).

FIG. 3 shows a Western blot performed to determine which fractions contain Fetuin-A using an antibody against human Fetuin-A. Fetuin-A was detected in the Load (L) and elution (A) fractions and slightly in the CIP (S) fraction but not in the flow though (FT). Fetuin-A standard (FetA), albumin standard (Alb).

FIG. 4 shows a Western blot performed to determine which fractions contain albumin using an antibody against human Albumin. Albumin was only detected in the Load (L) and CIP (S) fractions. Flow though (FT), elution (A) fractions, Fetuin-A standard (FetA), albumin standard (Alb).

FIG. 5 shows a Potency test of Fetuin-A in elongation of CPP formation in supersaturated calcium-phosphate solutions. The Fetuin-A concentrations in the legend are the concentration of Fetuin-A used to spike 0.079 g/L Fetuin-A in human serum. The assay shows that higher spiking concentrations of Fetuin-A purified by the method of the present invention delay the precipitation of calcium and phosphate. The triangles with solid lines indicate 0.375 g/L Fetuin-A, the squared dots with interrupted lines indicate 0.25 g/L Fetuin-A, the diamonds with solid lines indicate 0.125 g/L Fetuin-A, the circular dots with dotted lines indicate 0.079 g/L Fetuin-A, and the circular dots with dashed lines indicate 0.0625 g/L Fetuin-A,

FIG. 6 shows ovariectomized animals shows decreased bone mineral density (BMD) values compared to baseline values. Fetuin-A substitution shows improvement in the BMD (bone minerals density) values (100 and 400 ng/mL) similar to Bisphosphonate treated animals.

### EXAMPLES

### Example 1

### Method of Production of a Fetuin-A Preparation (Alpha-2-HS Glycoprotein) on Affi-Gel Blue Gel

For purification of Fetuin-A (alpha-2-HS glycoprotein) from a plasma derived preparation a Affi-Gel Blue Gel column (Bio-Rad Laboratories GmbH, Feldkirchen, Germany) comprising immobilized Cibacron® Blue F3GA dye was used, with a column volume (CV) of 68.4 mL. The chromatography was performed at 4° C.

### Running Conditions

Load: 910 mL (13.3 CV (column volumes)) feed paste IV (in 20 mM sodium phosphate pH 7.1)
Flow through 910 mL (13.3 CV)
Wash: 205.2 mL (3 CV)
Elution: 60 mL (1.5 CV) gradient
Eluate fractions: A16-A27 (A16-A24 were considered for eluate solution)

### Buffers in Order of Use

Equilibration and Wash buffer (buffer 1): 20 mM sodium phosphate, pH 7.1;
Gradient elution buffer (buffer 2): 20 mM sodium phosphate, pH 7.1 and 20 mM sodium phosphate+1.4 M NaCl pH 7.1, gradient total 1.5 CV; and
Regeneration buffer: 20 mM sodium phosphate+1.5 M KSCN, pH 7.1.
A gradient with increasing amount of buffer 2 is applied.

### Results

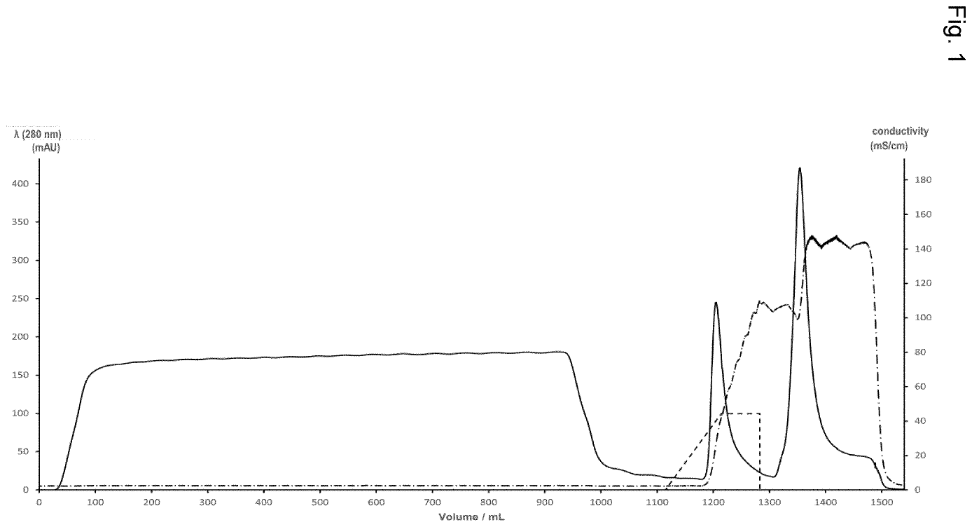

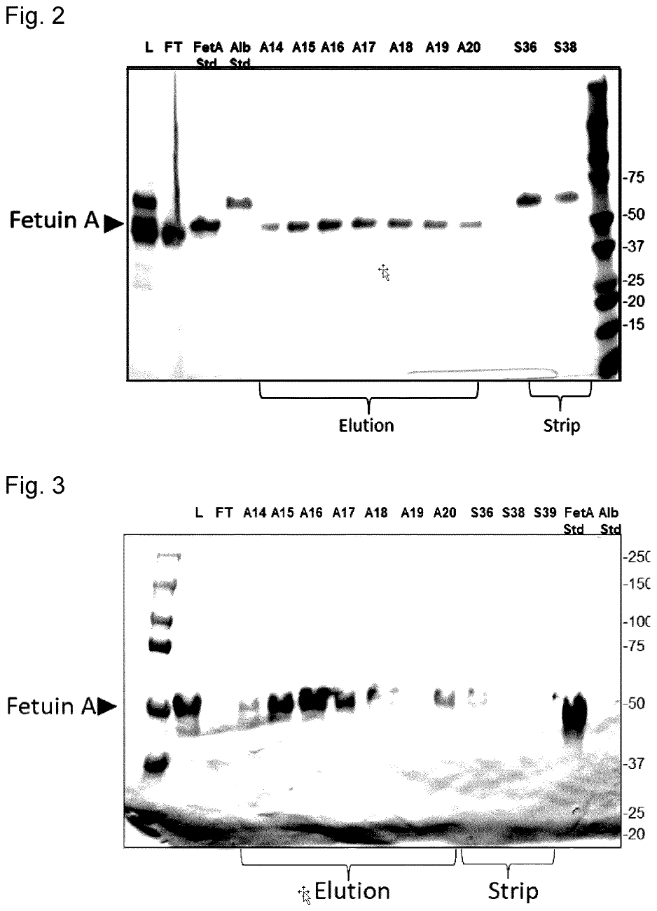

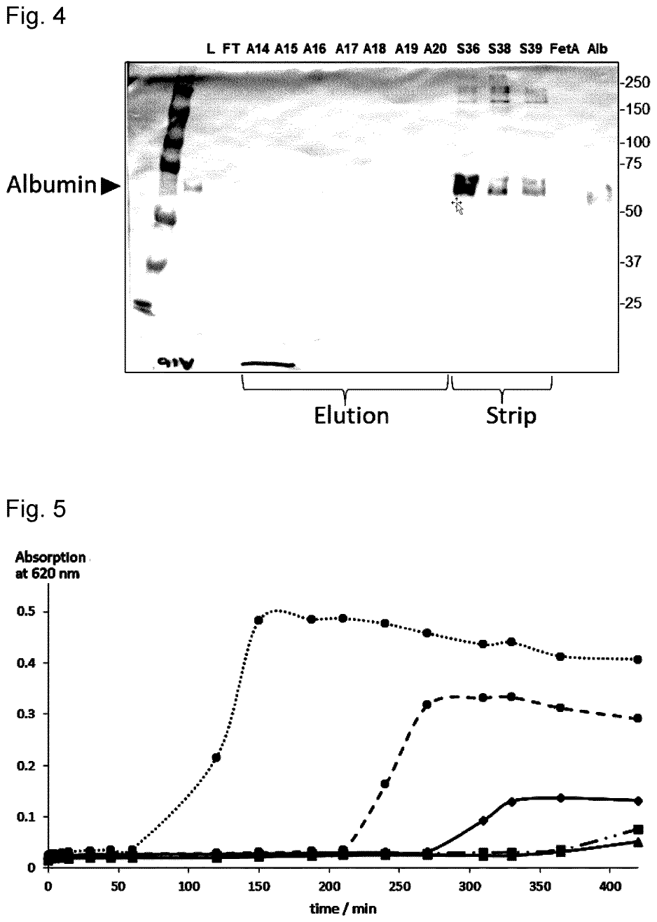

A resulting chromatogram is shown in FIG. 1. The x-axis indicates the volume in millilitres (mL), the left y-axis indicates the protein content as measured by OD280. The right y-axis indicates the conductivity in millisiemens per centimetre (mS/cm). Fetuin-A was found at a high purity in the eluted fractions A16-A24 (corresponding to volume 1190 mL-1232 mL). The chromatograms show as exemplified in FIG. 1, SDS-PAGE (reduced conditions) as shown in FIG. 2; and a Western Blot (reduced conditions) using Fetuin-A antibody (ABIN 2532784, Antibody-online, Aachen, Germany) of a chromatographic run in FIG. 3 and a Western Blot (reduced conditions) using albumin antibody of a chromatographic run of FIG. 4 demonstrate that the method is highly efficient for isolating Fetuin-A.

The inventors achieved good results for Fetuin-A activity by buffer exchange of the eluate protein solution using tangential flow filtration with Pall T-Serie TFF Cassette with Omega™ Membrane 200 cm² 30 kDa.

### Example 2

### Potency Testing for Purified Fetuin-A

To confirm the activity of Fetuin-A an assay using supersaturated calcium-phosphate solutions was developed to observe the elongation of CPP formation/calcium-phosphate precipitation over time. The assay was performed at 37° C. for 0-420 min in a 96-well plate.

### Buffers

Stock solution 1: calcium solution: 40 mM CaCl₂, 100 mM HEPES, 140 mM NaCl, pH 7.4 (adjusted with 6 M NaOH)
Stock solution 2: phosphate solution: 19.44 mM Na₂HPO₄, 4.56 mM NaH₂PO₄, 100 mM HEPES, 140 mM NaCl, pH 7.4 (adjusted with 6 M NaOH)

### Protein Solutions

Potency test was performed with albumin as a control in a concentration range of 10 g/L-100 g/L, Fetuin-A prepared in accordance with the present application in concentration range of 0.05 g/L-0.5 g/L, commercially available human recombinant Fetuin-A as reference in a concentration range of 0.05 g/L-0.5 g/L and human Fetuin-A in serum from donors.

### Results

Exemplary experimental results of the elongation of CPP formation caused by Fetuin-A (mature Fetuin-A in human Serum spiked with purified Fetuin-A from the inventor) are shown in FIG. 5. CPP formation can be detected by measuring the optical density. The results of the test showed at one hand that the purification method described above is very effective to produce active Fetuin-A from human plasma, and at the other hand that the purified Fetuin-A was as active as commercial recombinant Fetuin-A. The assay demonstrated the higher the spiking concentration of Fetuin-A (purified by PreviPharma) the more delayed was the precipitation of calcium and phosphate.

### Example 3

### Substitution of Fetuin-A in Osteoporosis Animal Model

Several challenges to understand the osteoporosis disease pathophysiology in humans are addressed by efficient animal models. To understand the postmenopausal osteoporosis, ovariectomy induced osteoporosis is well established and well accepted by scientific community. Rats are currently the most used laboratory animals to investigate osteoporosis. A well established bilateral ovariectomy model was used to investigate the efficacy of Fetuin-A substitution in the prevention of osteoporosis development. Six months old female Wistar rats underwent bilateral ovariectomy and allowed for 12 weeks to develop the disease. At 12 week time point, animals were randomized into several treatment groups (sham control group, Fetuin-A treatment group (with different doses) and animals for standard of treatment (Bisphosphonates and Monoclonal antibody treatment) (Table 1) and treatment began as according to the groups for another 8 weeks. At the of the study, animals were sacrificed according to ethical approval.

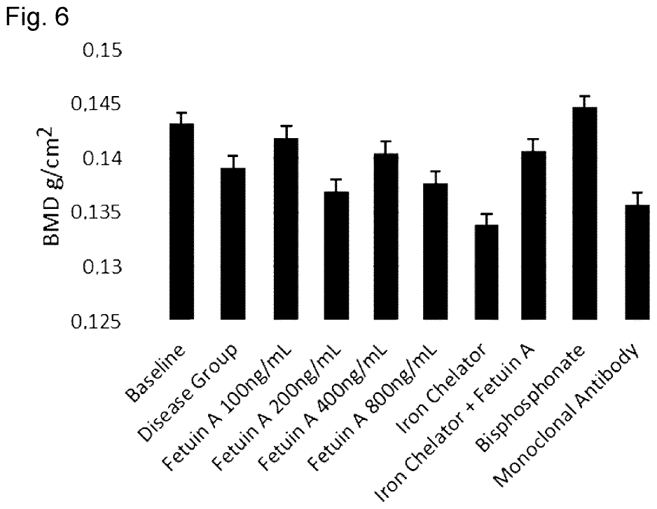

Osteoporosis is evaluated by examining the bone mineral density (BMD) using dual energy X-ray absorptiometry (DXA) and histopathological analysis as primary endpoint (FIG. 6). Serum parameters such levels of alkaline phosphatase, osteocalcin, RANKL, aspartate aminotransferase and alanine aminotransferase are measured as secondary evaluation points. Results are compared with baseline (before ovariectomy, week 0) vs study endpoint week 20.

TABLE 1
Various experimental control
groups and interventional doses

| | | | No. of |
|---|---|---|---|
| | | | injections/ |
| Group | Dose | | week |
| Control | — | | — |
| Fetuin-A | 100 | ng/ml | 1 |
| Bisphophonate | 1 | mg/kg BW | 3 |
| Monoclonal anti- | 1 | mg/kg BW | 2 |
| RANKL Antibody | | | |
| (Denosumab, | | | |
| Amgen GmbH, | | | |
| Munich, Germany) | | | |
| Iron chelator | 30 | mg/kg BW | 2 |

(Deferoxamin,
Novartis Pharma
GmbH, Nurnberg,
Germany)

### Examples 4

### Substitution of Fetuin-A in APMR, Caffey Disease and Osteogenesis Imperfecta—Bone Deformation Indications (APMR and Caffey Disease)

Childrens with alopecia mental retardation syndrome (APMR) have inborn Fetuin-A deficiency due to homozygous missense mutation in Alpha-2-Heremans-Schmid Glycoprotein (AHSG) gene (Reza Sailani M, Jahanbani F, Nasiri J, Behnam M, Salehi M et. al. Association of AHSG with alopecia and mental retardation (APMR) syndrome.

Hum Genet. 2017 March; 136(3):287-296) and studies by Muzammal and colleagues established the association of AHSG gene mutation and APMR disease phenotype (Muzammal M, Ahmad S, Ali M Z, Khan M A.: Alopecia-mental retardation syndrome: Molecular genetics of a rare neuro-dermal disorder. Ann Hum Genet. 2021 September; 85(5):147-154).

Further, children with infantile cortical hyperostosis which also known as Caffey Disease have in born Fetuin-A deficiency and deficiency is associated with the disease pathology (Merdler-Rabinowicz R, Grinberg A, Jacobson J M, Somekh I, Klein C, Lev A, Ihsan S, Habib A, Somech R, Simon A J: Fetuin-A deficiency is associated with infantile cortical hyperostosis (Caffey disease) Pediatr Res. 2019 November; 86(5):603-607).

Therefore, Fetuin-A can be used as therapeutic intervention in these patients.

1-15. (canceled)

16. A method for isolating Fetuin-A from a fluid containing Fetuin-A, wherein the method comprises the following steps:
(i) contacting the fluid containing Fetuin-A with a solid phase comprising a solid material and moieties of an anthraquinone compound;
(ii) allowing adhesion of the Fetuin-A to the solid phase;
(iii) at least partly removing the fluid from the solid phase and optionally washing the solid phase with a washing buffer; and
(iv) contacting the solid phase with an elution buffer and thereby eluting the Fetuin-A from the solid phase.

17. The method of claim 16, wherein the anthraquinone compound is Reactive Blue 2.

18. The method of claim 16, wherein the fluid is a body fluid.

19. The method of claim 17, wherein the body fluid is blood, blood serum, blood plasma, or a fraction thereof.

20. The method of claim 16, wherein the fluid is prepared from a blood plasma fraction selected from the group consisting of:
(a) crude V supernatant;
(b) paste IV or a subfraction thereof of the Cohn process;
(c) paste IV or a subfraction thereof of the Kistler-Nitschmann process;
(d) a waste fraction of paste I+II+III or I+III of the Cohn process or a combination thereof;
(e) a waste fraction of paste I+II+III or I+III of the Kistler-Nitschmann process or a combination thereof;
(f) paste I+II+III or paste I+III of the Cohn process or any fraction or a combination thereof;
(g) cryo-poor plasma supernatant or cryo-poor plasma precipitate;
(h) paste I+II+III or paste I+III of the Kistler-Nitschmann process or any fraction or a combination thereof; and
(i) a combination of two or more thereof.

21. The method of claim 16, wherein the solid phase comprises an affinity chromatography material.

22. The method of claim 21, wherein the solid phase comprises spherical beads of an affinity chromatography material.

23. The method of claim 16, wherein the anthraquinone compound is covalently conjugated to the solid material.

24. The method of claim 23, wherein the solid material is selected from the group consisting of a polysaccharide, a synthetic polymer, silica, and a combination of two or more thereof.

25. The method of claim 16, wherein the washing buffer is a buffer containing not more than 0.1 M of cations.

26. The method of claim 25, wherein the washing buffer is a buffer of pH 6 to 9 containing not more than 0.1 M of cations.

27. The method of claim 16, wherein the elution buffer is a buffer containing at least 0.5 M of cations.

28. The method of claim 27, wherein the elution buffer is a buffer of pH 6 to 9 containing more than 0.5 M of cations.

29. The method of claim 16, wherein the method further comprises one or more additional chromatographic steps.

30. The method of claim 16, wherein the method further comprises one or more of the following steps:
(a) subjecting a solution containing Fetuin-A to ultrafiltration/diafiltration, nanofiltration, a combination of ultrafiltration and nanofiltration, or any other virus removal procedure;
(b) a step of adjusting the pH of a solution containing the eluted Fetuin-A;
(c) a step of stabilizing the Fetuin-A by adding one or more stabilizers that prevent the Fetuin-A from degradation;
(d) one or more antiviral treatments, like nanofiltration, solvent/detergent treatment, pasteurization or dry-heat treatment
(e) adding one or more detergents;
(f) one or more detergents selected from the group consisting of: polyethylene glycol sorbitan monolaurate; polyethylene glycol sorbitan monooleate; polyoxyethylen(23)laurylether; polyethylene glycol hexadecyl ether, polyoxyethylene (20) cetyl ether; polyethylene glycol nonylphenyl ether; t-octylphenoxypolyethoxyethanol; octyl-phenoxy(polyoxyethylene)ethanol; nonyl-phenyl-polyethylene glycol; and derivatives thereof;
(g) adding one or more other antiviral agents;
(h) adding a phosphate ester as antiviral agent; or
(i) dialysis; or
(j) a combination of two or more thereof.

31. A composition comprising Fetuin-A obtainable from the method of claim 16, wherein the composition comprises at least 50% by weight, based on the total protein mass, of Fetuin-A.

32. Fetuin-A obtainable from the method of claim 16, for use as a medicament.

33. Fetuin-A obtainable from the method of claim 16, for use in a method for treating or preventing osteoporosis or being at risk of bone fractures, a risk for hypercalcification (calciphylaxis).

34. A method for determining the activity of Fetuin-A, the method comprising the following steps:
(i) providing a solution containing:
(a) a first solution containing soluble calcium salt,
(b) a second solution containing a soluble phosphate salt, and
(c) a sample containing the Fetuin-A;
(ii) mixing the components provided in step (i) with each other; and
(iii) determining the precipitation time of insoluble calcium phosphate,
wherein an elongated precipitation time indicates activity of Fetuin-A.
